(19) United States
(12) Patent Application Publication
(10) Pub. No.: US 2022/0183995 A1
(43) Pub. Date: Jun. 16, 2022

(54) SLEEP AID TRANSDERMAL PATCH AND ITS PROCESS OF PREPARATION

(71) Applicant: Azista Industries Pvt Ltd, Hyderabad (IN)

(72) Inventors: Srinivas Reddy Male, Hyderabad (IN); Praveen Kumar Mandapalli, Hyderabad (IN); Shantaram Laxman Pawar, Hyderabad (IN)

(21) Appl. No.: 17/442,523

(22) PCT Filed: Mar. 26, 2020
(86) PCT No.: PCT/IB2020/052842

§ 371 Date: Sep. 23, 2021

(30) Foreign Application Priority Data
Mar. 26, 2019 (IN) 201941011734

(51) Int. Cl. A61K 9/70; A61K 36/84; A61K 45/06

(52) U.S. Cl. CPC A61K 9/7053; A61K 45/06; A61K 36/84

(57) ABSTRACT

The present invention relates to composition of sleep aid transdermal patch comprising natural herbal ingredient as active ingredient, synergistic additives and pharmaceutically acceptable excipients. The present invention relates to composition of transdermal patch comprising valerian as natural herbal ingredient, skullcap, licorice, passionflower as synergistic additives and pharmaceutically acceptable excipients to aid sleep in insomnia conditions. The present invention also relates to an efficient process for the preparation of sleep aid transdermal patch by using hot-melt coating technique (HMC), comprising the steps of melting, mixing, coating, laminating and cutting.

### FIELD OF THE INVENTION

The present invention relates to composition of sleep aid transdermal patch comprising natural herbal ingredient as active ingredient, synergistic additives and pharmaceutically acceptable excipients.

The present invention relates to composition of transdermal patch comprising valerian as natural herbal ingredient, skullcap, licorice, passionflower as synergistic additives and pharmaceutically acceptable excipients.

The present invention also relates to composition of transdermal patch comprising valerian as natural herbal ingredient, skullcap, licorice, passionflower as synergistic additives and pharmaceutically acceptable excipients to aid sleep in insomnia conditions.

The present invention also relates to an efficient process for the preparation of sleep aid transdermal patch by using hot-melt coating technique (HMC), comprising the steps of melting, mixing, coating, laminating and cutting.

### BACKGROUND OF THE INVENTION

Insomnia is considered the most common sleep complaint in patients suffering from various chronic diseases. On the other hand, elder people and menopausal women often suffer from sleep disturbances (initiating or maintaining sleep), which affects daily activities and biological rhythm.

Most commonly used medications for insomnia, including antihistamines, chloral hydrate, barbiturates, tryptophan, and melatonin. Although benzodiazepines are known to be effective for insomnia, but the adverse effects associated with are tolerance, dependence, and morning sleepiness, which limits its use.

### Valerian Extract

The herbal medicine valerian, the dried root of the plant Valeriana officinalis L., has been used as a medicinal herb since at least the time of ancient Greece and Rome. Its phytotherapeutical properties were described by Hippocrates as sedative and anti-anxiety. Galen prescribed it as a remedy for insomnia. Related species of the Valerianaceae family were used in traditional Chinese and Indian Ayurvedic medicine.

Valerian extracts became popular in the United States and Europe in the mid-1800s and continued to be used by both physicians and the lay public until it was widely replaced by prescription sedative drugs. Other common uses include the treatment of headaches, anxiety, palpitations, irritable or spastic bowel, menstrual cramps, high blood pressure, epilepsy and childhood behavior problems and learning.

Use of valerian preparations for effective treatment of insomnia has gained lot of interest when compared to benzodiazepines. Extracts from the roots of valerian (Valeriana officinalis L., Valerianaceae) have long been used in alternative medicine for the treatment and prophylaxis of GABA-related disorders, preferably selected from the group consisting of anxiety, depression, restlessness and insomnia.

The biological activities of the plant are attributed to valeranone, valerenadiene and valerenic acid. These compounds are typically found in the plant roots and are derived from a larger class of chemicals known as sesquiterpenes. Valeranone may be derived from germacrene, while the biosynthetic precursor for valerenic acid is thought to be valerena-1,10-diene, also known as valerenadiene.

Valerenic acid (VA) is one of the many active compounds in valerian. Generally, stress can lower levels of gamma-aminobutyric acid (GABA), which in turn has been linked to anxiety and impaired, poor quality sleep. The valerenic acid in valerian root has been shown to inhibit breakdown of GABA, resulting in greater calm and relaxation. The anti-anxiety potential of VA has been verified by in-vitro experiments on the gamma-aminobutyric acid type A receptor (GABAA) as well as by in-vivo experiments in mice and rats.

Valerian is often combined with other sedating herbs, such as hops (Humulus lupulus) and lemon balm (Melissa officianalis), to treat insomnia.

### Passionflower

Passionflower (Passiflora incarnate) may help to treat anxiety and insomnia. The main active constituents are flavonoids (vitexin). It appears to boost the level of gamma-aminobutyric acid (GABA) in brain. This compound lowers brain activity, which may help to relax and sleep better.

Often passion flower is combined with other natural sleep remedies and sold as over the counter (OTC) treatments for anxiety, insomnia and other sleep disorders. It's common to find these natural sleep aids containing a combination of valerian root, lemon balm, St. John's Wort, chamomile and lavender flower.

Passionflower is used as a component in sedatives (combined with valerian root and lemon balm), a pediatric sedative tea (Species nervinae pro infantibus: with lemon balm, lavender flower, and St John's Wort) and cardiotonic formulations (with hawthorn). It is also a German homeopathic medicine for pain, insomnia, and neurasthenia. Passionflower is commonly a component of herbal sleep aid formulations.

Passionflower extracts consist of fresh or dried aerial parts of P. incarnate or P. alata, collected during the flowering and fruiting period. Extracts contain 0.825% apigenin and luteolin glycosides, vitexin, isovitexin and their C-glycosides, kaempferol, quercetin, and rutin; indole alkaloids (0.01%), mainly harman, harmaline, harmine; coumarin derivatives; cyanogenic glycosides (gynocardin); amino acids (including GABA); fatty acids (linoleic and linolenic); gum; maltol; phytosterols (stigmasterol); sugars (sucrose); and a trace of volatile oil.

Numerous pharmacological effects of Passiflora incarnata are mediated via modulation of the GABA system including affinity to GABA (A) and GABA (B) receptors, and effects on GABA uptake. Although the active ingredients have not been conclusively delineated, most available data suggests flavonoids and GABA account for the reported effects.

### Skullcap

Skullcap (Scutellaria baicalensis) is well known for its anxiolytic properties. It is often used as a sleep aid by itself or mixed together with other calming herbs such as valerian. The main active ingredient present in skullcap is baicalein and it has shown anxiolytic properties due to binding to the benzodiazepine binding site of the GABAA receptor.

Baicalin possessed anti-hyperglycemic activities by suppressing hepatic gluconeogenesis. Baicalein could reduce endometriosis by suppressing the viability of human endometrial stromal cells in vitro. Furthermore, baicalin exhibited embryo-protection, weight losing, sleep-wake regulation, antiallergic, and anti-pyretic effects. The polysaccharides from S. baicalensis showed antioxidative and immunostimulating activities.

Scutellaria baicalensis contains at least 126 small molecules and 6 polysaccharides. It possesses anti-tumor, anti-viral, anti-microbial, anti-inflammatory, antioxidative and neuroprotective activities.

### Licorice

The licorice (Glycyrrhiza glabra) is well known for its use for long term stress and adrenal gland fatigue, which is often the result of stress. The main component of the roots is glycyrrhizin. Chronic fatigue, irritation and exhaustion are a sign of the daily stress that puts pressure on your life and a sign that something in your body may be out of tune. As it acts on the adrenal glands, it may increase your resistance to stress and be helpful with a midlife crisis, as this depressive state may be caused by adrenal exhaustion. Glycyrrhiza glabra, is known to induce sleep and even increase sleep duration.

### Hot-Melt Coating Technology

In recent years, use of hot melt adhesives plays important role in transdermal drug delivery devices due to their superior physico-chemical properties. In solvent based technique mostly adhesives like PIB, acrylate, silicone derivatives are being used which controls the drug release by functional group interaction between drug and adhesive. Since herbal extracts contain high amount of oily substances, which tend to show cold flow property which in turn lead to less tack and poor adherence to skin when patches formulated using solvent-based coating technique. On the other hand, drugs that are not stable in aqueous matrix cannot be formulated by water based hydrogel technique.

In addition, Hot-melt coating technology has several advantages; firstly, HMC is economical and relatively faster process compared to solvent or water-based coatings. Secondly, HMC is particularly suitable for combined adhesive/drug-matrix device as they can be formulated to contain little or no chemical functional group. This reduces the possibility of medication/adhesive interactions and skin irritation. Thirdly, hot melt adhesive is less prone to swelling when in contact with alcohols used in certain medications, which is a particular problem with acrylics.

U.S. Pat. No. 6,190,689 B1 discloses hydrophilic pressure sensitive hot-melt adhesives which comprises vegetable preparations, e.g., extracts or tinctures. Vegetable preparations in hydrophilic pressure sensitive hot-melt adhesives according to the present invention may also be used in the transdermal therapy, for example, ginseng extract in case of geriatric complaints; valerian tincture, extracts of melissa and hop to cause a sedative effect in case of super excitation, sleep disturbances and stress.

U.S. Pat. No. 6,448,303 B1 discloses hot melt pressure sensitive adhesive especially suited for adhesive skin application, including transdermal drug delivery applications. The adhesive is used as a carrier contact adhesive or overlay contact adhesive for transdermal patches.

U.S. Pat. No. 6,869,622 B2 pharmaceutical composition for improving sleep quality, and more specifically relates to a pharmaceutical composition comprising pharmaceutically effective amounts of an extract of the plant Valeriana officinalis L. and methods of using such compositions to improve sleep quality, as measured via a variety of specific criteria.

U.S. Pat. No. 7,064,242 B2 discloses a patch comprising a backing layer and a adhesive layer formed on the back face of the backing layer wherein a woven fabric or a nonwoven fabric made of a rayon and pulp fiber mixture is employed as the backing layer and the mixing ratio thereof is from 3:7 to 7:3. It also discloses valerian extract as a plant extract.

U.S. Pat. No. 8,512,769 B2 discloses valerian root (radix Valeriana officinalis) has long been employed in conditions of unrest as well as anxiety-produced sleep-onset insomnia.

U.S. Pat. No. 9,375,463 B2 discloses composition consisting of lavender, valerian, hops, ashwagandha, melatonin, magnesium, vitamin B12 is combined with additional ingredients, devil's claw, bromelain and boswellia extract, for the purpose of addressing symptoms including but not limited to insomnia associated with pain. The herb-based composition can be used alone or further formulated with pharmaceutically acceptable compounds, vehicles, or adjuvants with a favorable delivery profile, i.e., suitable for delivery to a subject.

WO Publication No. 2008/122100 A1 discloses that valerian (Valeriana officinalis) is a perennial plant native to North America, Asia and Europe. The root has been traditionally used as treatment for anxiety and insomnia.

WO Publication No. 2013/143843 A1 discloses valerian extract for the treatment and prophylaxis of GABA-related disorders, preferably GABAA-related disorders, more preferably selected from the group consisting of anxiety, depression, restlessness and insomnia, wherein the ratio of valerenic acid (VA) to acetoxyvalerenic acid (AVA) in the extract is at least 2:1.

WO Publication No. 2016/174011 A1 discloses medicament comprising valerian extracts or their derivatives. It also discloses composition comprising extracts from plants belonging to the genus Valeriana, preferably Valeriana officinalis, or active ingredient derivatives, in particular valeric acid or valerenic acid. It also discloses valerian is a commonly used herbal medicinal product for the treatment of anxiety and insomnia.

There are several sleep aid transdermal patches available in the market like Sleep ZPatch which contains valerian, passion flower, skullcap, hops, 1-theanine, melatonin, 5-HTP, GABA and cosmoperine; Sleep Nirvana sleep aid patch which contains melatonin, hops, passion flower and valerian; Sleep starter topical patch which contains magnesium, valerian, hops, 5-HTP and melatonin. Sleep aid transdermal patches comprising all the four herbal ingredients valerian, passion flower, skullcap and licorice are not available in the market.

All the prior art references related to the use of valerian tincture in preparing hydrophilic pressure sensitive hot melt adhesive, valerian use in treating anxiety, depression, restlessness, insomnia and use of valerian in combination with sedating herbs in treating insomnia. However, the inventors of present invention provide composition of sleep aid transdermal patch using valerian as active ingredient and skull cap, licorice, passion flower as synergistic additives and pharmaceutically acceptable excipients. The inventors of present invention also provide an efficient process for the preparation of sleep aid transdermal patch of valerian using hot-melt coating (HMC) technology comprising the steps of melting, mixing, coating, laminating and cutting.

### OBJECTIVE OF INVENTION

The main objective of the present invention is to provide a composition of sleep aid transdermal patch comprising natural herbal ingredient as active ingredient, synergistic additives and pharmaceutically acceptable excipients.

Another objective of the present invention is to provide sleep aid transdermal patch composition comprising valerian as natural herbal ingredient, skullcap, licorice, passionflower as synergistic additives and permeation enhancer, hot melt adhesives, tackifier, vehicle as pharmaceutically acceptable excipients to aid sleep in insomnia conditions.

Another objective of the present invention is to provide composition of sleep aid transdermal patch comprising valerian as active ingredient, skullcap, licorice, passionflower as synergistic additives, oleic acid as permeation enhancers, (styrene isoprene thermoplastic elastomer)-SIS 5002 and pressen 1471 as hot melt adhesives, arkon-P 100 as tackifier and mineral oil as vehicle.

Still another objective of the present invention is to provide a process for the preparation of sleep aid transdermal patch by using hot-melt coating technique (HMC) comprising the steps of melting, mixing, coating, laminating and cutting.

Still another objective of the present invention is to provide process for the preparation of sleep aid transdermal patch comprising steps of melting hot melt adhesives under stirring, adding vehicle and tackifier to molten adhesive, adding active ingredient, synergistic additives along with permeation enhancer to form hot molten base under stirring, coating, laminating and cutting into desired size.

Still another objective of the present invention is to provide an improved/efficient manufacturing process for preparation of transdermal patch by hot-melt coating technique, which is a solvent-free technique, faster and more economic coating process. In addition, HMC technique has several advantages including drug release, adhesiveness (tack) and physical properties of patch can be tuned relatively easy compared to solvent based coating technique.

In yet another objective of the present invention is to provide sleep in insomnia conditions by transdermal application of valerian patch.

### SUMMARY OF INVENTION

Accordingly, the present invention provides composition of sleep aid transdermal patch useful in facilitating sleep and relief in insomnia conditions.

In one embodiment, the present invention provides composition of sleep aid transdermal patch comprising natural herbal ingredient as active ingredient, synergistic additives and pharmaceutically acceptable excipients.

In another embodiment, the present invention provides composition of sleep aid transdermal patch comprising valerian as natural herbal ingredient, skullcap, licorice, passionflower as synergistic additives and permeation enhancer, hot melt adhesives, tackifier, vehicle as pharmaceutically acceptable excipients.

In another embodiment, the present invention is to provide composition of sleep aid transdermal patch comprising valerian as active ingredient and skullcap, licorice, passionflower as synergistic additives and permeation enhancer, hot melt adhesives, tackifier, vehicle as pharmaceutically acceptable excipients to aid sleep in insomnia conditions.

In another embodiment, the present invention provide composition of sleep aid transdermal patch comprising valerian as active ingredient and skullcap, licorice, passionflower as synergistic additives, oleic acid as permeation enhancer, (styrene isoprene thermoplastic elastomer)-SIS 5002 and pressen 1471 as hot melt adhesives, arkon-P 100 as tackifier and mineral oil as vehicle.

In another embodiment, the present invention provides process for the preparation of sleep aid transdermal patch by using hot-melt coating technique (HMC) comprising the steps of melting, mixing, coating, laminating and cutting.

In yet another embodiment, the present invention provides a sleep aid transdermal patch composition comprising:
0.1% to 5% (w/w) of active ingredient,
0.1% to 10% (w/w) of synergistic additives,
0.1% to 3% (w/w) of permeation enhancer,
20 to 90% (w/w) of hot melt adhesives,
1 to 10% (w/w) of tackifier, and
0.5 to 20% (w/w) of vehicle.

In yet another embodiment, the present invention provides a sleep aid transdermal patch composition of valerian comprising:
0.1% to 5% (w/w) of valerian,
0.1% to 10% (w/w) of synergistic additives (passionflower, skullcap and licorice),
0.1% to 3% (w/w) of oleic acid,
20 to 90% (w/w) of SIS 5002 and pressen 1471,
1 to 10% (w/w) of arkon-P 100, and
0.5 to 20% (w/w) of mineral oil.

In still another embodiment, the present invention provides an improved/efficient manufacturing process for preparation of transdermal patch by hot-melt coating technique, which is a solvent-free technique, faster and more economic coating process. In addition, HMC technique has several advantages including drug release, adhesiveness (tack) and physical properties of patch can be tuned relatively easy compared to solvent based coating technique.

In yet another embodiment of the present invention provides a process for preparing sleep aid transdermal patch, the process comprising steps of:
a) melting hot melt adhesives under stirring at 100-180° C.,
b) adding vehicle and tackifier to molten adhesive under stirring at 100-180° C.,
c) adding active ingredient and synergistic additives along with permeation enhancer to molten adhesive blend under stirring to obtain homogenous material,
d) coating on to the polyethylene terephthalate release liner,
e) laminating the obtained adhesive matrix, and
f) cutting into desired size to get transdermal patch, pouching and labelling.

In yet another embodiment of the present invention provides a process for preparing valerian sleep aid transdermal patch, the process comprising steps of:
a) melting SIS 5002 and Pressen 1471 under stirring at 100-180° C.,
b) adding mineral oil and arkon-P 100 to molten adhesive under stirring at 100-180° C.,
c) adding valerian and passionflower, skullcap, licorice along with oleic acid to molten adhesive blend under stirring to obtain homogenous material,
d) coating on to the polyethylene terephthalate release liner,
e) laminating the obtained adhesive matrix, and
f) cutting into desired size to get transdermal patch, pouching and labelling.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

The present invention provides sleep aid transdermal patch composition of sleep aid transdermal patch comprising natural herbal as active ingredient, synergistic additives and pharmaceutically acceptable excipients.

The present invention provides composition of sleep aid transdermal patch comprising valerian as natural herbal, skullcap, licorice, passionflower as synergistic additives and permeation enhancer, hot melt adhesives, tackifier, vehicle as pharmaceutically acceptable excipients.

The present invention provides composition of sleep aid transdermal patch comprising valerian as active ingredient and skullcap, licorice, passionflower as synergistic additives, oleic acid as permeation enhancer, (styrene isoprene thermoplastic elastomer)-SIS 5002 and pressen 1471 as hot melt adhesives, arkon-P 100 as tackifier and mineral oil as vehicle.

The term “active ingredients” of the present invention is used to relieve a person in need from insomnia conditions. Preferably used active ingredient is valerian.

Valerian has been used in sleeplessness with anxiety, spasms with mental tension, pain with muscle tension, anti convulsive for tremors, spasms, palpitations, menstrual cramps, eclectic use in cases of depression, nervous debility, as a stimulating nervine, headaches, palpitations, irritable or spastic bowel, high blood pressure, epilepsy and childhood behavior problems and learning.

The extract of the root of valerian (Valeriana officinalis), a flowering plant, has been widely used to treat sleeping disorders. Valerian is an effective treatment for insomnia, it may be an important treatment alternative because it is relatively inexpensive and without known side effects.

The valerenic acid in valerian root has been shown to inhibit breakdown of GABA, resulting in greater calm and relaxation. Provides sedative and sleep-enhancing properties. Restores circadian rhythm and maintains daytime freshness. Safe choice in case of stress-related conditions that result in sleeplessness, anxiety and irritability.

The concentration of valerian used in the sleep aid transdermal patch is in the range of 0.1 to 5% (w/w), more preferably 0.75 to 1.875% (w/w) of the total weight of the composition.

Valerian when used in combination with synergistic additives including passion flower, licorice and skullcap can provide synergistic additive effect in providing better sleep in insomnia conditions.

The concentration of synergistic additives used in the sleep aid transdermal patch is in the range of 0.1 to 10% (w/w). Preferably used concentration of synergistic additives individually is from 0.125 to 0.75% (w/w). The concentration of synergistic additives used in combination in the sleep aid transdermal patch of the present invention is from 0.1 to 3% (w/w).

The term “hot melt adhesives” of the present invention includes combination of one or more hot melt adhesives and includes at least two adhesives selected from the group of ethylene-vinyl acetate copolymer series (EVA hot melt adhesive), synthetic rubber-based hot melt adhesives, polyolefin based hot melt adhesive, polyamide based hot melt adhesive, polyester-based hot melt adhesives, polyurethane-based hot melt adhesives, styrene isoprene thermoplastic elastomer. Preferably, hot melt adhesives are (styrene isoprene thermoplastic elastomer)-SIS 5002 and pressen 1471.

Hot melt adhesives used in the sleep aid transdermal patch is in the range of 20 to 90% (w/w). Preferably used concentration of hot melt adhesives individually is from 23 to 60% (w/w). The concentration of hot melt adhesives used in combination in the sleep aid transdermal patch of the present invention is from 70 to 90% (w/w), more preferably in the range of 82 to 83.125% (w/w) of the total weight of the composition.

Permeation enhancer used in the composition of the present invention include, but are not limited to azones, isopropylmyristate, fatty acids, menthol, essential oils, terpenes, terpenoids, N-methyl-2-pyrrolidone, 1-dodecyl-azacycloheptan-2-one, oleic acid, oleyl alcohol, linoleic acid, isopropyl linoleate, butanediol, lauryl alcohol, lauryl acetate, lauryl lactate, ethyl acetate, dimethyl isosorbide, isostearic acid. Preferably, the permeation enhancer is oleic acid.

Permeation enhancer used in the sleep aid transdermal patch is in the range of 0.1 to 3% (w/w), more preferably in the range of 0.1 to 2% of the total weight of the composition.

Tackifier used in the composition of the present invention include, but are not limited to petroleum resins (e.g., aliphatic hydrocarbon resins, alicyclic hydrocarbon resins, and aromatic hydrocarbon resins), phenolic resins, xylene resins and coumarone indene resins rosin derivatives (e.g., rosin, glycerin esters of rosin, hydrogenated rosins, glycerin esters of hydrogenated rosin, pentaerythritol esters of rosin, etc.), saturated alicyclic hydrocarbon resins (e.g., ARKON P-100), aliphatic hydrocarbon resins (e.g., Quintone B170) terpene resins (e.g., Clearon P-125), maleic acid resins and the like. Preferably, the tackifier is arkon-P 100.

Tackifier used in the sleep aid transdermal patch is in the range of 1 to 10% (w/w), more preferably in the range of 3 to 6% (w/w) of the total weight of the composition.

“Vehicle” as used herein refer to carrier materials suitable for transdermal drug administration, and include any such materials known in the art, i.e., any liquid gel, solvent, liquid diluent, adhesive, or the like, which is nontoxic and which does not interact with other components of the composition in a deleterious manner. Vehicle, which also may function as solvents in some instances, are used to provide the compositions of the invention in their preferred form. Examples include, but not limited to, water, ethanol, propanol, isopropanol, mineral oil, silicone, polyethylene glycol, polypropylene glycol, liquid sugars, waxes, petroleum jelly and a variety of other oils, aloe and polymeric materials along with polyacrylate, silicone, natural and synthetic rubbers or other adhesives. Preferably, the vehicle is mineral oil.

Vehicle used in the sleep aid transdermal patch is in the range of 0.5 to 20% (w/w), more preferably in the range of 5 to 15% (w/w) of the total weight of the composition.

The transdermal patch of the present invention has been prepared by hot melt coating technique. The advantage of this technique is simple and easy to manufacture, more economical and solvent free technique. Using HMC technique tuning drug release, adhesiveness (tack) and physical properties of patch is relatively good compared to solvent-based coating technique.

The transdermal patch of the present invention has been prepared by hot melt coating technique using polyethylene terephthalate release liner and coated layer is laminated using nonwoven or woven fabric backing material.

The molten adhesive blend preparation comprising the SIS and pressen 1471 as hot melt adhesives. The physical & mechanical properties of adhesive matrix are achieved only with the combination of SIS 5002 and Pressen 1471 to get the desired adhesion & flexibility to adhesive matrix.

The content of SIS and pressen 1471 should contain 20% to 90% by mass with respect to total mass of transdermal patch. If the content falls within this range, the cohesive property and tack of adhesive layer can be maintained. Accordingly, favorable application properties can be obtained.

For the transdermal patch preparation of present invention, it is preferred that the permeation enhancer used to enhance the permeation of active ingredient and synergistic additives through the skin to provide better and fast therapeutic action. The concentration of permeation enhancer should be in the range of 0.1-3% by mass with respect to total mass of the adhesive matrix.

For the transdermal patch preparation of present invention, it is preferred that the tackifier play a key role to maintain optimum sticking to skin. The physical and mechanical properties of transdermal patch are achieved only with the optimized concentration of tackifier to get the desired tackiness & flexibility. The concentration of tackifier should be in the range of 1-10% by mass with respect to total mass of adhesive matrix.

Various properties associated with each component of the transdermal patch compositions may affect the properties of the final product. Properties associated with the selection of raw materials, molecular weight, concentration and viscosity may influence the adhesive matrix formation, adhesion and therapeutic effect.

The invention disclosed herein is process for the preparation of transdermal patch useful in facilitating sleep.

### Manufacturing Process for Sleep Aid Transdermal Patch:

### 1. Preparation of Hot Melt Adhesive Blend

The hot melt adhesive blend is prepared by melting of SIS and pressen 1471 under stirring preferably at 100° C.-180° C. temperature. Later, add mineral oil and arkon-P 100 to the molten adhesive under stirring to obtain homogenous adhesive blend. Preferably, the concentration of SIS should be in the range of 5-30% (w/w) and pressen 1471 should be in the range of 20-80% (w/w), preferably, mineral oil should be in the range of 0.5-20% (w/w), preferably, arkon-P 100 should be in the range of 1-10% (w/w).

### 2. Addition of Active and Synergistic Additives

Add valerian, passionflower, skullcap and licorice along with oleic acid to the molten adhesive blend under stirring to obtain homogenous material. Preferably, the concentration of valerian should be in the range of 0.1-5% (w/w), preferably, the concentration of passionflower should be in the range of 0.1-2% (w/w), preferably, the concentration of skullcap should be in the range of 0.1-2% (w/w), preferably, the concentration of licorice should be in the range of 0.05-2% (w/w), preferably, the concentration of oleic acid should be in the range of 0.1-3% (w/w).

### 3. Coating

The hot melt adhesive blend was uniformly coated with desired thickness on to the polyethylene terephthalate release liner.

### 4. Lamination and Cutting and Packaging

Then coated adhesive matrix was laminated using Nonwoven fabric.

### 5. Cutting and Packaging

Then the resulting product cut into a desired size to produce transdermal patch and finally packed in triple laminated aluminum pouch.

Formulations were developed using different concentrations of oleic acid, SIS, pressen 1471 and arkon-P 100. The formulations prepared with different variations were evaluated for their description, adhesion (tack), peel test, assay.

The following examples describes the nature of the invention and are given only for the purpose of illustrating the present invention in more detail and are not limitative and relate to solutions, which have been particularly effective on bench scale.

### Example 1

| | | Concentration | mg/ |
|---|---|---|---|
| S.No. | Ingredients | (%) | patch |
| 1. | Valerian (Valeriana officinalis) | 0.75 | 3 |
| 2. | Passionflower (Passiflora incarnata) | 0.75 | 3 |
| 3. | Skullcap (Scutellaria baicalensis) | 0.25 | 1 |
| 4. | Licorice (Glycyrrhiza glabra) | 0.125 | 0.5 |
| 5. | Oleic acid | 1 | 4 |
| 6. | (Styrene Isoprene thermoplastic | 23.125 | 92.5 |
| | elastomer)-SIS 5002 | | |
| 7. | Pressen 1471 | 60 | 240 |
| 8. | (Alicyclic saturated hydrocarbon | 5 | 20 |
| | resin)-Arkon-P 100 | | |
| 9. | Mineral oil | 9 | 36 |

### Example 2

| | | Concentration | mg/ |
|---|---|---|---|
| S.No. | Ingredients | (%) | patch |
| 1. | Valerian (Valeriana officinalis) | 1.125 | 4.5 |
| 2. | Passionflower (Passiflora incarnata) | 0.75 | 3 |
| 3. | Skullcap (Scutellaria baicalensis) | 0.25 | 1 |
| 4. | Licorice (Glycyrrhiza glabra) | 0.125 | 0.5 |
| 5. | Oleic acid | 1 | 4 |
| 6. | (Styrene Isoprene thermoplastic | 23 | 92 |
| | elastomer)-SIS 5002 | | |
| 7. | Pressen 1471 | 59.75 | 239 |
| 8. | (Alicyclic saturated hydrocarbon | 5 | 20 |
| | resin)-Arkon-P 100 | | |
| 9. | Mineral oil | 9 | 36 |

### Example 3

| | | Concentration | mg/ |
|---|---|---|---|
| S.No. | Ingredients | (%) | patch |
| 1. | Valerian (Valeriana officinalis) | 1.5 | 6 |
| 2. | Passionflower (Passiflora incarnata) | 0.75 | 3 |
| 3. | Skullcap (Scutellaria baicalensis) | 0.25 | 1 |
| 4. | Licorice (Glycyrrhiza glabra) | 0.125 | 0.5 |
| 5. | Oleic acid | 1 | 4 |
| 6. | (Styrene Isoprene thermoplastic | 23 | 92 |
| | elastomer)-SIS 5002 | | |
| 7. | Pressen 1471 | 59.375 | 237.5 |
| 8. | Alicyclic saturated hydrocarbon | 5 | 20 |
| | resin)-Arkon-P 100 | | |
| 9. | Mineral oil | 9 | 36 |

### Example 4

| | | Concentration | mg/ |
|---|---|---|---|
| S.No. | Ingredients | (%) | patch |
| 1. | Valerian (Valeriana officinalis) | 1.875 | 7.5 |
| 2. | Passionflower (Passiflora incarnata) | 0.75 | 3 |
| 3. | Skullcap (Scutellaria baicalensis) | 0.25 | 1 |
| 4. | Licorice (Glycyrrhiza glabra) | 0.125 | 0.5 |
| 5. | Oleic acid | 1 | 4 |
| 6. | (Styrene Isoprene thermoplastic | 23 | 92 |
| | elastomer)-SIS 5002 | | |
| 7. | Pressen 1471 | 59 | 236 |
| 8. | Alicyclic saturated hydrocarbon | 5 | 20 |
| | resin)-Arkon-P 100 | | |
| 9. | Mineral oil | 9 | 36 |

### Manufacturing Process:

Required quantity of pressen 1471 and SIS, were molten in hot vessel at 100° C.-180° C. under stirring.

Required quantity of arkon and mineral oil were added to molten adhesive blend under stirring at 100° C.-180° C. temperature to obtain homogenous blend.

Required quantity of all herbal extracts, valerian, skullcap, licorice and passion flower along with oleic acid were added to above molten adhesive base under stirring to obtain homogenous mixture of active ingredient and synergistic additives in blend for coating.

Sleep aid transdermal patch prepared as per the present invention is evaluated at different stability conditions and the data is given as below:

TABLE 1
Stability Condition: 40° C./75% RH for
transdermal patch of Example 3

| Test | Specification | Initial | 3 Month | 6 Month |
|---|---|---|---|---|
| Description | Brown to dark brown | Complies | Complies | Complies |
| | coloured matrix type | | | |
| | transdermal patch that | | | |
| | is square shape patch | | | |
| Assay | Each patch | 100.1 | 100.2 | 99.5 |
| (Total | contains 6 mg of | | | |
| Valerenic | Valerenic acids. | | | |
| acid) | Limit: (5.4 mg | | | |
| | to 6.6 mg) | | | |
| | i.e 90% to 110% | | | |
| | of label claim | | | |
| Uniformity of | L1 ≤ 15 | 3.1 | 3.8 | 4.2 |
| Dosage Units | | | | |
| (by Content | | | | |
| Uniformity) | | | | |
| Tack Test (N) | 2.5-6.0 | 4.2 | 4.0 | 3.8 |
| Peel Test | 1.5-4.0 | 2.7 | 2.4 | 2.2 |

Microbial Enumeration Limit @

| | | | | |
|---|---|---|---|---|
| Total Aerobic | Not more than 100 | Absent | NA | Absent |
| Microbial | | | | |
| Count (cfu/gm) | | | | |
| Total yeasts | Not more than 10 | Absent | NA | Absent |
| and molds | | | | |
| count (cfu/gm) | | | | |
| Pseudomonas | Absent | Absent | NA | Absent |
| aeruginosa | | | | |
| (gm) | | | | |
| Staphylococcus | Absent | Absent | NA | Absent |
| aureus | | | | |
| (gm) | | | | |

TABLE 2
Stability Condition: 30° C./75% RH for
transdermal patch of Example 3

| Test | Specification | Initial | 3 Month | 6 Month |
|---|---|---|---|---|
| Description | Brown to dark brown | Complies | Complies | Complies |
| | coloured matrix type | | | |
| | transdermal patch that | | | |
| | is square shape patch | | | |
| Assay | Each patch | 100.1 | 99.8 | 99.7 |
| (Total | contains 6 mg of | | | |
| Valerenic | Valerenic acids. | | | |
| acid) | Limit: (5.4 mg to | | | |
| | 6.6 mg) i.e 90% | | | |
| | to 110% of label | | | |
| | claim | | | |
| Uniformity of | L1 ≤ 15 | 3.1 | 3.6 | 3.8 |
| Dosage Units | | | | |
| (by Content | | | | |
| Uniformity) | | | | |
| Tack Test | 2.5-6.0 | 4.2 | 3.8 | 3.7 |
| Peel Test | 1.5-4.0 | 2.7 | 2.3 | 2.2 |

Microbial Enumeration Limit @

| | | | | |
|---|---|---|---|---|
| Total Aerobic | Not more than 100 | Absent | NA | Absent |
| Microbial | | | | |
| Count (cfu/gm) | | | | |
| Total yeasts | Not more than 10 | Absent | NA | Absent |
| and molds | | | | |
| count (cfu/gm) | | | | |
| Pseudomonas | Absent | Absent | NA | Absent |
| aeruginosa | | | | |
| (gm) | | | | |
| Staphylococcus | Absent | Absent | NA | Absent |
| aureus (gm) | | | | |

TABLE 3
Stability Condition: 25° C./60% RH for
transdermal patch of Example 3

| Test | Specification | Initial | 3 Month | 6 Month |
|---|---|---|---|---|
| Description | Brown to dark brown | Complies | Complies | Complies |
| | coloured matrix type | | | |
| | transdermal patch that | | | |
| | is square shape patch | | | |
| Assay | Each patch | 100.1 | 100 | 100.2 |
| (Total | contains 6 mg of | | | |
| Valerenic | Valerenic acids. | | | |
| acid) | Limit: (5.4 mg to | | | |
| | 6.6 mg) i.e 90% | | | |
| | to 110% of label | | | |
| | claim | | | |
| Uniformity of | L1 ≤ 15 | 3.1 | 3.2 | 3.5 |
| Dosage Units | | | | |
| (by Content | | | | |
| Uniformity) | | | | |
| Tack Test | 2.5-6.0 | 4.2 | 4.1 | 3.9 |
| Peel Test | 1.5-4.0 | 2.7 | 2.6 | 2.4 |

Microbial Enumeration Limit @

| | | | | |
|---|---|---|---|---|
| Total Aerobic | Not more than 100 | Absent | NA | Absent |
| Microbial | | | | |
| Count (cfu/gm) | | | | |
| Total yeasts | Not more than 10 | Absent | NA | Absent |
| and molds | | | | |
| count (cfu/gm) | | | | |
| Pseudomonas | Absent | Absent | NA | Absent |
| aeruginosa | | | | |
| (gm) | | | | |
| Staphylococcus | Absent | Absent | NA | Absent |
| aureus (gm) | | | | |

We claim:

1. A sleep aid transdermal patch composition comprising Valerian as herbal active ingredient, synergistic additives and pharmaceutically acceptable excipients.

2. The sleep aid transdermal patch composition as claimed in claim 1 comprising:
(a) Valerian,
(b) synergistic additives,
(c) permeation enhancer,
(d) hot melt adhesives,
(e) tackifier, and
(f) vehicle.

3. The sleep aid transdermal patch composition as claimed in claim 2, wherein the Valerian is in the range of 0.1 to 5% (w/w), more preferably 0.75 to 1.875% (w/w) of the total weight of the composition.

4. The sleep aid transdermal patch composition as claimed in claim 1, wherein the synergistic additives are skullcap, licorice and passionflower used individually or in combination.

5. The sleep aid transdermal patch composition as claimed in claim 4, wherein the individual synergistic additive is in the range of 0.1 to 10% (w/w), preferably used concentration of synergistic additives individually is from 0.125 to 0.75% (w/w) of the total weight of the composition.

6. The sleep aid transdermal patch composition as claimed in claim 4, wherein the combination synergistic additives are in the range of 0.1 to 3% (w/w) of the total weight of the composition.

7. The sleep aid transdermal patch composition as claimed in claim 2, wherein the permeation enhancer is selected from azones, isopropylmyristate, fatty acids, menthol, essential oils, terpenes, terpenoids, N-methyl-2-pyrrolidone, 1-dodecyl-azacycloheptan-2-one, oleic acid, oleyl alcohol, linoleic acid, isopropyl linoleate, butanediol, lauryl alcohol, lauryl acetate, lauryl lactate, ethyl acetate, dimethyl isosorbide and isostearic acid.

8. The sleep aid transdermal patch composition as claimed in claim 7, wherein the permeation enhancer is in the range of 0.1 to 3% (w/w), more preferably in the range of 0.1 to 2% of the total weight of the composition.

9. The sleep aid transdermal patch composition as claimed in claim 2, wherein the hot melt adhesives includes at least two adhesives selected from group of ethylene-vinyl acetate copolymer series (EVA hot melt adhesive), synthetic rubber-based hot melt adhesives, polyolefin based hot melt adhesive, polyamide based hot melt adhesive, polyester-based hot melt adhesives, polyurethane-based hot melt adhesives and styrene isoprene thermoplastic elastomer.

10. The sleep aid transdermal patch composition as claimed in claim 9, wherein the hot melt adhesives is a combination of (styrene isoprene thermoplastic elastomer)-SIS 5002 and pressen 1471.

11. The sleep aid transdermal patch composition as claimed in claim 9, wherein the hot melt adhesives are in the range of 20 to 90% (w/w), preferably used concentration of hot melt adhesives individually is from 23 to 60% (w/w), preferably used in combination in the range of 70 to 90% (w/w), more preferably used combination in the range of 82 to 83.125% (w/w) of the total weight of the composition.

12. The sleep aid transdermal patch composition as claimed in claim 2, wherein the tackifier is selected from petroleum resins (e.g., aliphatic hydrocarbon resins, alicyclic hydrocarbon resins, and aromatic hydrocarbon resins), phenolic resins, xylene resins and coumarone indene resins rosin derivatives (e.g., rosin, glycerin esters of rosin, hydrogenated rosins, glycerin esters of hydrogenated rosin, pentaerythritol esters of rosin, etc.), saturated alicyclic hydrocarbon resins (e.g., ARKON P-100), aliphatic hydrocarbon resins (e.g., Quintone B170) terpene resins (e.g., Clearon P-125) and maleic acid resins.

13. The sleep aid transdermal patch composition as claimed in claim 12, wherein the tackifier is in the range of 1 to 10% (w/w), more preferably in the range of 3 to 6% (w/w) of the total weight of the composition.

14. The sleep aid transdermal patch composition as claimed in claim 2, wherein the vehicle is selected from water, ethanol, propanol, isopropanol, mineral oil, silicone, polyethylene glycol, polypropylene glycol, liquid sugars, waxes, petroleum jelly, variety of other oils, aloe, polymeric materials along with polyacrylate, silicone, natural and synthetic rubbers or other adhesives.

15. The sleep aid transdermal patch composition as claimed in claim 14, wherein the vehicle is in the range of 0.5 to 20% (w/w), more preferably in the range of 5 to 15% (w/w) of the total weight of the composition.

16. A process for preparing sleep aid transdermal patch claimed in claim 1, wherein the process comprising steps of:
(a) melting hot melt adhesives under stirring at 100-180° C.,
(b) adding vehicle and tackifier to molten adhesive under stirring at 100-180° C.,
(c) adding active ingredient and synergistic additives along with permeation enhancer to molten adhesive blend under stirring to obtain homogenous material,
(d) coating on to the polyethylene terephthalate release liner,
(e) laminating the obtained adhesive matrix, and
(f) cutting into desired size to get transdermal patch, pouching and labelling.

17. The process for preparing sleep aid transdermal patch as claimed in claim 16, wherein the process comprising steps of:
(a) melting SIS 5002 and Pressen 1471 under stirring at 100-180° C.,
(b) adding mineral oil and arkon-P 100 to molten adhesive under stirring at 100-180° C.,
(c) adding Valerian and passionflower, skullcap, licorice along with oleic acid to molten adhesive blend under stirring to obtain homogenous material,
(d) coating on to the polyethylene terephthalate release liner,
(e) laminating the obtained adhesive matrix, and
(f) cutting into desired size to get transdermal patch, pouching and labelling.
